(19) United States
(12) Patent Application Publication
(10) Pub. No.: US 2005/0267480 A1
(43) Pub. Date: Dec. 1, 2005

(54) Spinal facet fixation device

(76) Inventor: Loubert Suddaby, Orchard Park, NY (US)

Correspondence Address: SHOEMAKER AND MATTARE, LTD, 10 POST OFFICE ROAD - SUITE 110, SILVER SPRING, MD, 20910, US

(21) Appl. No.: 11/181,785

(22) Filed: Jul. 15, 2005

Related U.S. Application Data
(63) Continuation of application No. 10/230,140, filed on Aug. 29, 2002.

(51) Int. Cl. A61B 17/58

(52) U.S. Cl. 606/75

(57) ABSTRACT

A spinal facet fixation device includes a pair of jaws hinged on a common base. A threaded actuator bears against a cam surface on the jaws to draw the points of the jaws together. A surgeon applies the opposed points to respective facets of vertebral elements, and then tightens the actuator to draw the facets together.

This is a continuation of application Ser. No. 10/230140, filed Aug. 29, 2002.

### BACKGROUND OF THE INVENTION

This invention relates to the field of orthopedic spinal fusion surgery and particularly to the process of interarticular facet fixation or fusion, serving to stabilize adjacent vertebral elements, thereby facilitating the development of bony union between them and thus long term spinal stability.

Of all animals possessing a backbone, human beings are the only creatures who remain upright for significant periods of time. From an evolutionary standpoint, this erect posture has conferred a number of strategic benefits, not the least of which is freeing the upper limbs for purposes other than locomotion. From an anthropologic standpoint, it is also evident that this unique evolutionary adaption is a relatively recent change and as such has not benefitted from natural selection as much as have backbones held in the horizontal attitude. As a result, the stresses acting upon the human backbone (or “vertebral column”), are unique in many senses, and result in a variety of problems or disease states that are peculiar to the human species.

The human vertebral column is essentially a tower of bones held upright by fibrous bands called ligaments and contractile elements called muscles. There are seven bones in the neck or cervical region, twelve in the chest or thoracic region, and five in the low back or lumbar region. There are also five bones in the pelvis or sacral region which are normally fused together and form the back part of the pelvis. This column of bones is critical for protecting the delicate spinal cord and nerves, and for providing structural support for the entire body.

Between the vertebral bones themselves exist soft tissue structures—discs—composed of fibrous tissue and cartilage which are compressible and at as shock absorbers for sudden downward forces on the upright column. More importantly, the discs allow the bones to move independently of each other to permit functional mobility of the column of spinal vertebrae. Unfortunately, the repetitive forces which act on these intervertebral discs during repetitive day-to-day activities of bending, lifting and twisting cause them to break down or degenerate over time.

Presumably because of humans' posture, their intervertebral discs have a high propensity to degenerate. Overt trauma, or covert trauma occurring in the course of repetitive activities disproportionately affect more highly mobile areas of the spine. Disruption of a disc's internal architecture leads to bulging, herniation or protrusion of pieces of the disc and eventual disc space collapse. Resulting mechanical and even chemical irritation of surrounding neural elements (spinal cord and nerves) cause pain, attended by varying degrees of disability. In addition, loss of disc space height relaxes tension on the longitudinal spinal ligaments, thereby contributing to varying degrees of spinal instability.

While various types of spinal fusion operations have been developed, most procedures involving the articular facets have focused either on the passive grafting of bone between facet surfaces denuded of their synovium, or mechanical fixation of the facet joint with a simple screw. In the former case, additional instrumented fixation of the spine is required to prevent dislodgement of the bone grafts from between the articular surfaces of the joint and in the latter case, the procedure is largely adjunctive since its long term success is usually dependent upon bony union occurring elsewhere between the adjacent vertebral elements being fused, i.e., interbody or inter-transverse postero-lateral fusions.

### SUMMARY OF THE INVENTION

An object of this invention to provide for a facet fixation device that can be utilized either directly in a stand alone facet fusion procedure or as an adjunctive fixator to be utilized when other forms of spinal fusion are employed, e.g., as back up for an anterior fusion. It is also the object of this invention to provide for deployment of the device either radiographically or through endoscopically assisted minimally invasive approaches.

To achieve these objectives, the invention provides a device having opposable jaws bearing teeth or pointed tips to grasp, clasp, crimp or hold the articular surfaces of a single facet joint, thereby immobilizing the joint. The resultant inhibition of mobility serves to facilitate bony union or fusion of the involved spinal elements either directly at the facet joint or at some other chose point between the involved vertebral segments.

The clasping action of the opposable jaws is achieved by a screw- or ratchet-type mechanism that allows for varying degrees of opposition while simultaneously inhibiting unwarranted or undesirable separation or expansion of component elements. In the simplest version, a metal structure approximating the function of a staple can be crimped together to achieve fixation of the facet joint.

In primary facet fusion, the device is to be applied after a wafer of bone has been placed between the articular surfaces of the facet joint suitably prepared by decortication. As an adjunctive fixator, the device may be applied radiographically or endoscopically to an intact facet joint thereby inhibiting movement at the joint site until fusion is achieved elsewhere.

In either scenario, the salient feature of the device is the opposable nature of the component elements that function as jaws to bite and hold the separate articular components of the facet joint thereby serving to immobilize them. The jaws of the device in turn have pointed tips or teeth that engage the cortical surfaces of the joint as the jaws are mechanically closed. Unidirectional closure of the fixation device is achieved either through a screw or a ratchet mechanism which prevents opening of the jaws or disengagement of the teeth once the desired degree of crimping has been achieved.

### BRIEF DESCRIPTION OF THE DRAWINGS

In the accompanying drawings,

FIG. 1 is a top plan view of a spinal facet fixation device embodying the invention;

FIG. 2 is a front elevation thereof; and

FIG. 3 is a side elevation thereof.

FIG. 4 is a top plan view of a second form of the invention;

FIG. 5 is a front elevation thereof; and

FIG. 6 is a side elevation thereof.

FIG. 7 is a top plan view of a third form of the invention;

FIG. 8 is a front elevation thereof; and

FIG. 9 is a side elevation thereof.

FIGS. 10 and 11 show a three-finger version of the device shown in FIGS. 1-3.

FIGS. 12 and 13 show the device of FIG. 1 being applied to hold superior and inferior articular facets together.

FIGS. 14 and 15 show, respectively, a spinal facet staple, and the staple being crimped over a pair of spinal facets.

### DESCRIPTION OF THE PREFERRED EMBODIMENT

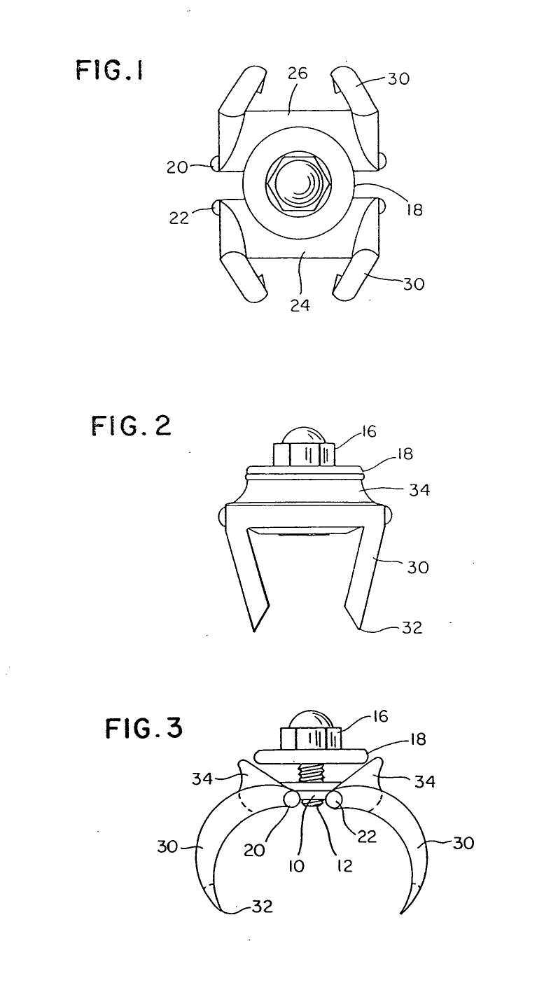

The spinal facet fixation device shown in FIGS. 1-3 comprises a base member 10 having a threaded post 12 affixed at its center and extending perpendicularly therefrom. A nut 16 having an integral or captive washer 18 thereon is threaded onto the post.

A pair of pivot pins 20,22 are affixed to the bottom of the base, equally offset from the center. The ends of the pins fit within holes (not shown) formed in respective jaws 24,26. Each of the jaws has one or more curved fingers 30, each terminating at a pointed tip 32.

The upper surface of each jaw has an upwardly protruding cam 34 (FIG. 3) designed to bear against the washer. When the nut 18 is turned clockwise, it advances down the post, and the washer 16, bearing against the cams 34 on either side, forces the jaws to pivot downward, bringing their pointed tips 32 closer together.

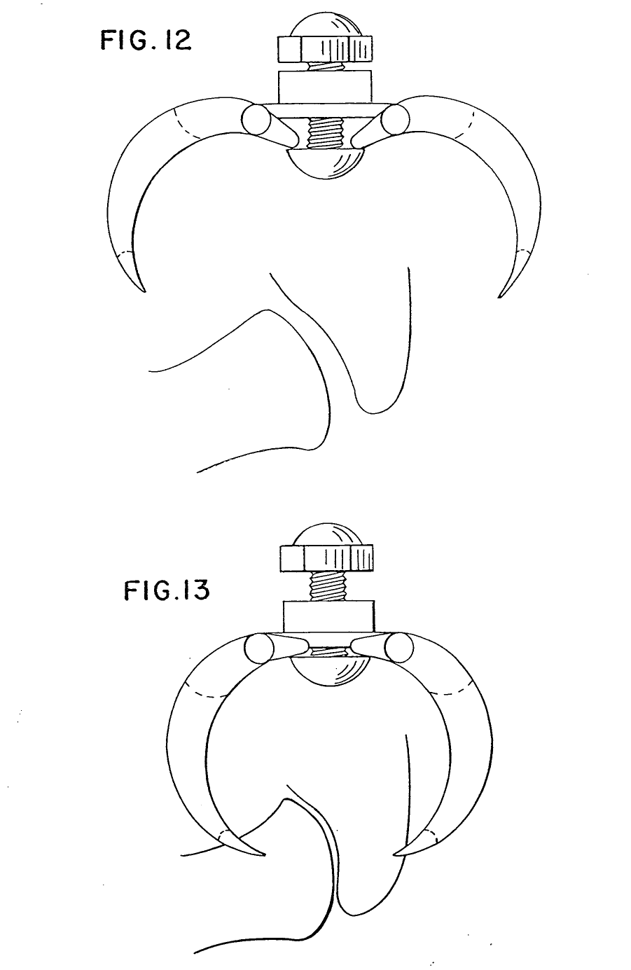

To draw a facet joint together, a surgeon places the pointed tips of the jaws against neighboring facets (FIG. 12), and then tightens the nut, whereupon the jaws draw the facets more closely together (FIG. 13) and retain them thus.

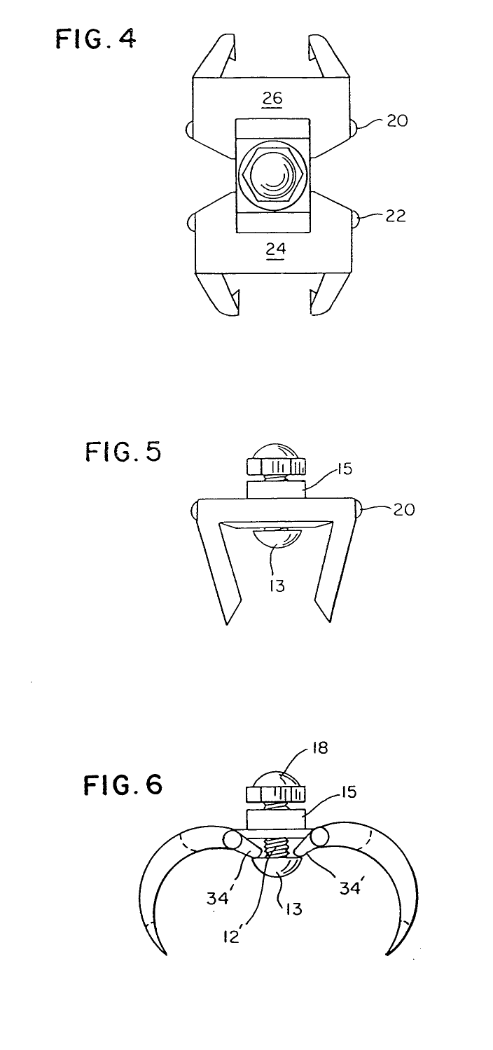

An alternative form of the invention is shown in FIGS. 4-6. Here, the jaws have inwardly extending cam followers 34′ rather than the upwardly protruding cam surface 34 of the first embodiment. The post 12′ in this instance has a rounded head 13 at its bottom; its threaded shaft extends through a threaded collar 15 on the base. The threads may be left-handed, if desired, in which case the head moves upward when the nut us turned clockwise, raising the cam followers and levering the fingers downward to grasp the facets. Other variations on the details of the actuating mechanism will occur to those of ordinary skill.

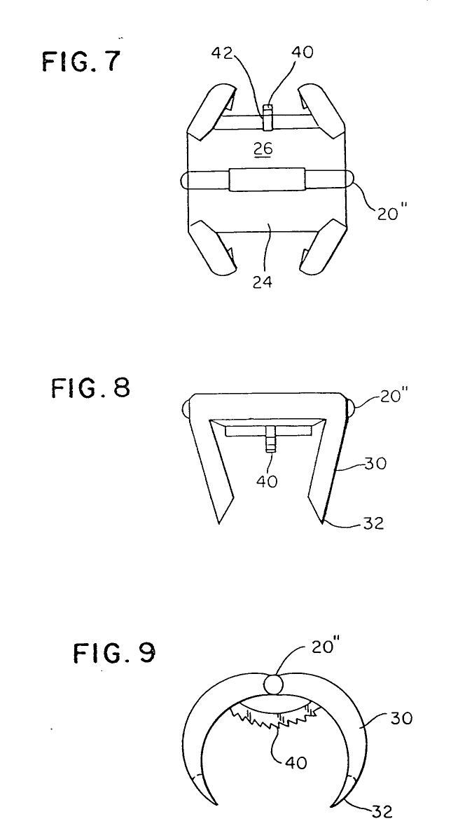

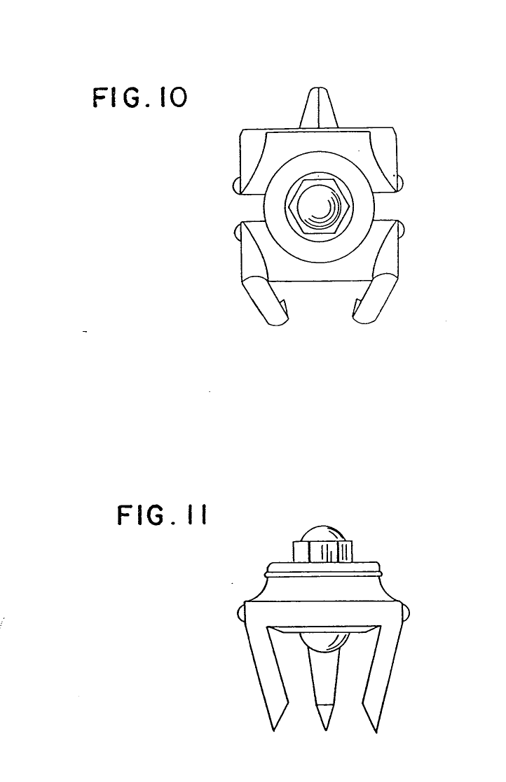

A third form of the invention is shown in FIGS. 7-9. In this case, the jaws are hinged on along an axis by a single pin 20″. The threaded actuator and cams have been replaced by a ratchet segment 40 having raked teeth which permit the jaws to be drawn together, but does not permit them to spread apart thereafter. One end of the segment is fixed to the jaw 24; the other passes through a slot 42 on the jaw 26 so that its teeth catch against the side of the slot. This type of device is closed with a tool such as forceps.

The number of fingers on each jaw may be varied, depending on the intended application. In the examples illustrated in FIGS. 1-9, each jaw each had two fingers. As an exemplary variation, FIGS. 10 and 11 show the device of FIGS. 1- 3, modified to have only one finger on one of the jaws, two fingers on the other. The exact shape of the fingers, and the geometry of their tips, may also be selected according to preference and intended use.

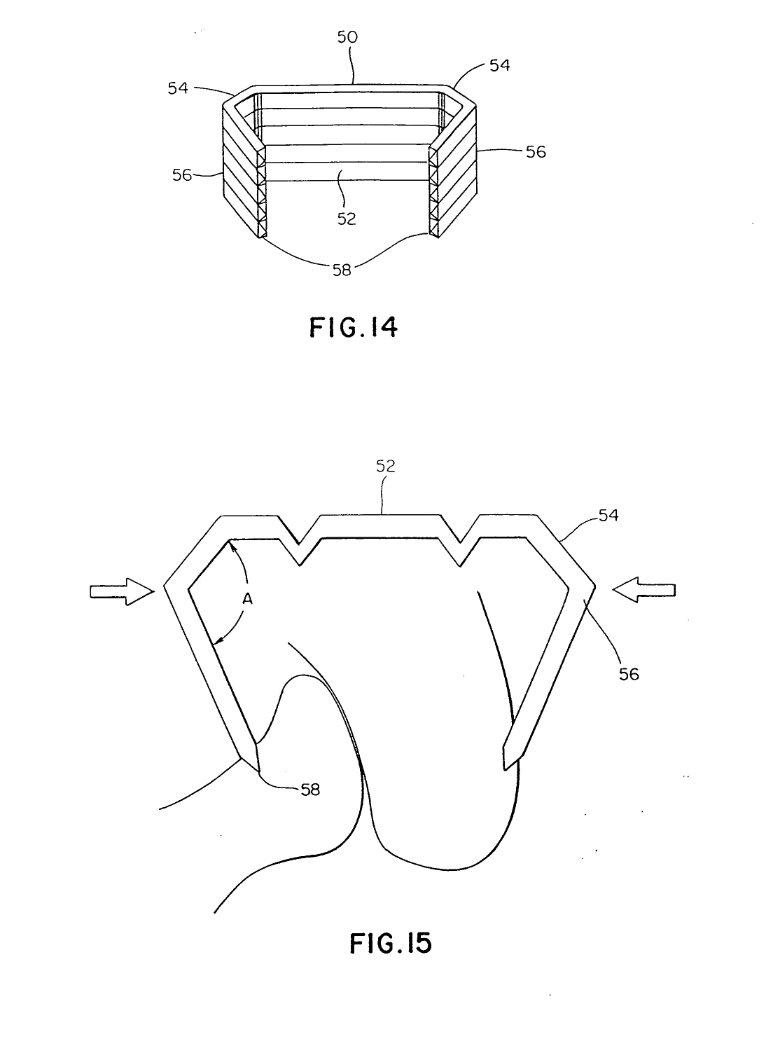

The invention can also be practiced with a spinal facet staple 50, illustrated in FIGS. 14 and 15. The staple has a center portion 52 extending between opposed arms 54, each of which has an elbow 56 subtending an obtuse angle A. The obtuse angles face one another so that the tips 58 are directed along axes which, if extended, would intersect. In use, the staple is placed with a suitable crimping tool (not shown) so that the tips engage neighboring spinal facets. Then the tool is manipulated to apply sufficient inward force to the elbows to crimp (permanently deform) the center portion as illustrated in FIG. 15, drawing the facets toward one another, and holding them together after the tool is released.

Since the invention is subject to modifications and variations, it is intended that the foregoing description and the accompanying drawings shall be interpreted as only illustrative of the invention defined by the following claims.

1. A method of fixing adjacent facets of a spine, comprising steps of
introducing into a space around said facets a tool having a pair of opposed hinged jaws and a mechanism for drawing the jaws together, and
closing the jaws around the facets.

2. A spinal facet fixation clamp for use in fusing adjacent vertebrae of a spine, said clamp comprising
a base,
a pair of opposed jaws having hinge connections to the base,
each said jaw comprising at least one finger terminating at a pointed tip, the fingers being curved toward one another, and
a mechanism for closing the jaws so that the tips grasp and fixate a pair of spinal facets.

3. The invention of claim 2, wherein at least one of said jaws has at least two curved fingers, so that the clamp makes at least three point contact with the spine.

4. The invention of claim 3, wherein each jaw has at least two curved fingers.

5. The invention of claim 2, wherein the closing mechanism comprises a cam.

6. The invention of claim 5, wherein each jaw has a cam surface thereon, and the closing mechanism further comprises a threaded actuator having a bearing surface which bears against each cam surface and closes the jaws when turned in one direction.

7. The invention of claim 7, wherein said threaded actuator comprises a nut, and said bearing surface is on said nut.
